(19) United States
(12) Patent Application Publication
(10) Pub. No.: US 2015/0099916 A1
(43) Pub. Date: Apr. 9, 2015

(54) HEAD IMMOBILIZATION DEVICE WITH INHALER, METHOD FOR MAKING SAME AND METHOD OF USING SAME

(71) Applicant: Lori Linn JUREK, Kent, WA (US)

(72) Inventor: Lori Linn JUREK, Kent, WA (US)

(21) Appl. No.: 14/048,407

(22) Filed: Oct. 8, 2013

(51) Int. Cl. A61N 5/10; B29C 51/00; A61M 16/12; A61M 16/00; A61M 15/00; A61M 16/10

(52) U.S. Cl. CPC A61N 5/10; A61M 15/0086; A61M 16/104; A61M 16/12; A61M 16/009; B29C 51/00; A61N 2005/1097; A61M 16/01
USPC 600/1; 128/845; 264/271.1

(57) ABSTRACT

Head and neck radiation therapy for the treatment of cancer or the like is carried out using a mask which comprises a heat-softenable, conformable and perforate plastic sheet and a frame which can be secured to the surface of a treatment table or chair to immobilize the patient's head and neck. An inhaler is placed on the patient's face prior to molding the thermoplastic sheet over the patient's face such that the resulting contoured sheet captures the inhaler and produces a unitary assembly. Inlet and outlet hoses can be attached to the inhaler for administering an anesthetic or soporific in gaseous form, such as nitrous oxide, for the purpose of calming and tranquilizing the patient for reducing feelings of claustrophobic anxiety during the treatment. A separate scavenging unit may be added.

### FIELD OF THE INVENTION

This disclosure relates to an apparatus for immobilizing a portion of a patient's body, such as the head and neck, during a treatment process while, at the same time, administering an anesthetic or sedative in gaseous form. The disclosure also relates to a method for making the apparatus and a method for using the apparatus in the course of a treatment such as radiation therapy.

### BACKGROUND OF THE INVENTION

It is known to use a mask-like device to immobilize a patient's head and upper body during an oncological treatment such as radiation therapy where precise aiming of the radiation source relative to a cancer site is essential. One such immobilizing device, available from Klarity Medical Products of Newark, Ohio, comprises a U-shaped frame which can be secured to a treatment table and a thermoplastic net secured to the frame. The thermoplastic net is originally flat with small openings, but is molded to the facial contours of the patient for use in treatment. The thermoplastic net is heated to soften it sufficiently to assume the contours of a patient's face and head while remaining attached to the frame. This is done by gently laying the heat-softened net over the patient's face and head. When cooled, the contours become permanent. The contoured device is typically labeled in such a way as to personalize it to a specific patient, and is stored at the therapy facility for use during each subsequent radiation therapy session. There are similar devices large enough to cover not only the head and face, but much of the upper body as well. The same preparation steps are used for it.

A common problem which occurs during such a treatment is that the immobilization device creates feelings of anxiety, similar to or based on claustrophobia and/or merinthophobia, which feelings can range from mild to severe. When added to the anxieties often associated with the basic treatment, the result is significant but unnecessary stress and a dread of the treatment sessions. This can cause a patient to delay or prematurely terminate treatment.

### SUMMARY OF THE INVENTIONS

A basic aspect of our invention is the enhancement of a radiation therapy procedure involving an immobilization device using a patient-conforming mask as described above. The enhancement involves combining the immobilization device with an inhaler such that a gaseous anesthetic or sedative, such as nitrous oxide, may be administered to the patient prior to and/or during the therapy procedure. When the anesthetic is administered prior to and/or during the radiation therapy procedure, the result is a significant reduction in stress and a diminution of the anxieties associated with the immobilization experience.

Another aspect of the invention described herein is the construction of the immobilization device. This aspect is hereinafter described with reference to illustrative embodiments wherein the immobilization device comprises a frame that can be secured to a treatment table or head support, a body-contoured perforate mask sheet which can be placed conformingly over the patient's face, head and neck, and an anesthetic inhaler located in the mask in a position to cover the patient's nose. The mask is described herein as an illustrative example is made of a perforate sheet material without contours; i.e., essentially flat. Combining the mask and inhaler may be carried out by placing the inhaler on the patient or on a model and thereafter heating and molding a perforate, thermoplastic sheet over the inhaler and the patient's face or the face of a model. In most cases, the inhaler is of the type having tubes connectable to a gaseous anesthetic source and/or a scavenging system as hereinafter described with reference to an illustrative embodiment. An exemplary inhaler described herein is made of an elastomeric material such as rubber and is available from Accutron, Inc. of Phoenix, Ariz.

A third aspect of the disclosure is a method of carrying out a radiation therapy session comprising the steps of:
a. immobilizing a portion of a patient's body such as the head and neck using a conforming perforate mask;
b. administering a gaseous anesthetic or sedative, such as nitrous oxide, to the patient using an inhaler associated with the mask; and
c. administering radiation to the patient while the head and neck remain immobilized and the gaseous anesthetic is being administered.

The term “anesthetic” as used herein is essentially synonymous with “inhalant”, “sedative”, and “soporific” and may, in addition to or as an alternative to nitrous oxide, involve one or more of horocaine, ether, chloroform, cocaine, sodium pentothal, chloral hydrate, morphine, procaine, Canadol, and scopolamine, as long as the anesthetic/inhalant/soporific is in gaseous form. The anesthetic acts as a calmative or sedative or soporific, but typically does not induce sleep.

The various features and advantages of our inventions and the various aspects thereof will be best understood from reading the following specification which is to be taken with the accompanying drawings.

### BRIEF SUMMARY OF THE DRAWINGS

The description herein makes reference to the accompanying drawings wherein like reference numerals refer to like parts throughout the several views and wherein:

FIG. 1 is a perspective view of an immobilizing mask associated with an underlying inhaler;

FIG. 2 is an exploded view of the mask of FIG. 1 showing the inhaler in perspective;

FIG. 3 is a perspective view of a step in the process of preparing the immobilizing mask;

FIG. 4 is a perspective view of another step in the process of preparing the mask;

FIG. 5 shows a technician placing the heat-softened mask over a patient or model with an inhaler in place;

FIG. 6 shows a technician creating openings in the conformed mask for inhaler ports;

FIG. 7 is a top perspective view of the fully conformed immobilizer device;

FIG. 8 is a perspective view of the immobilizer with anesthetic supply tubes attached; and

FIG. 9 is a perspective view of the immobilizer with a scavenging appliance added.

### DETAILED DESCRIPTION OF THE ILLUSTRATIVE EMBODIMENTS AND METHODS

### The Device

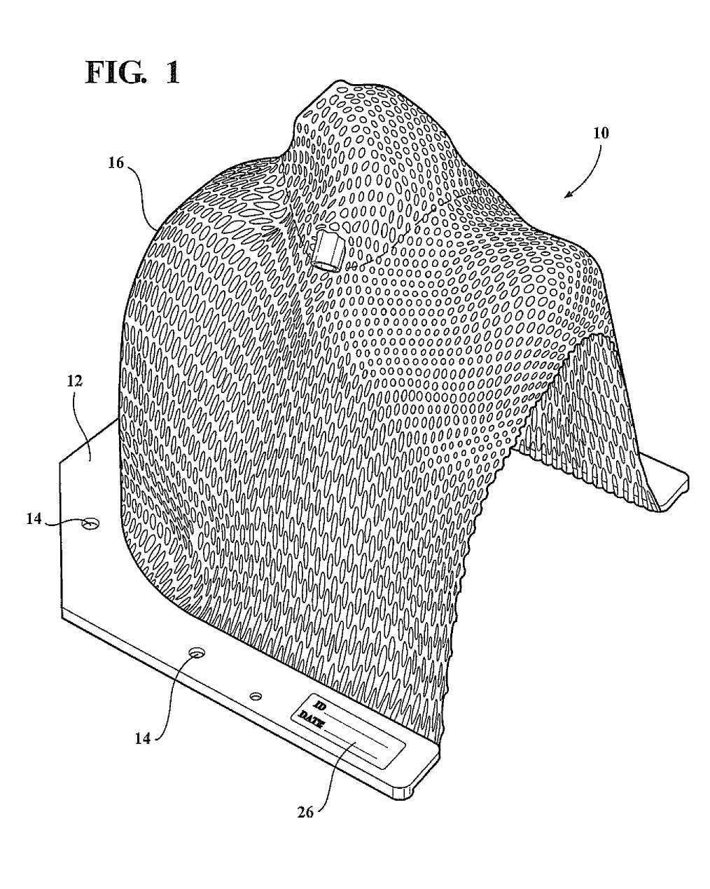

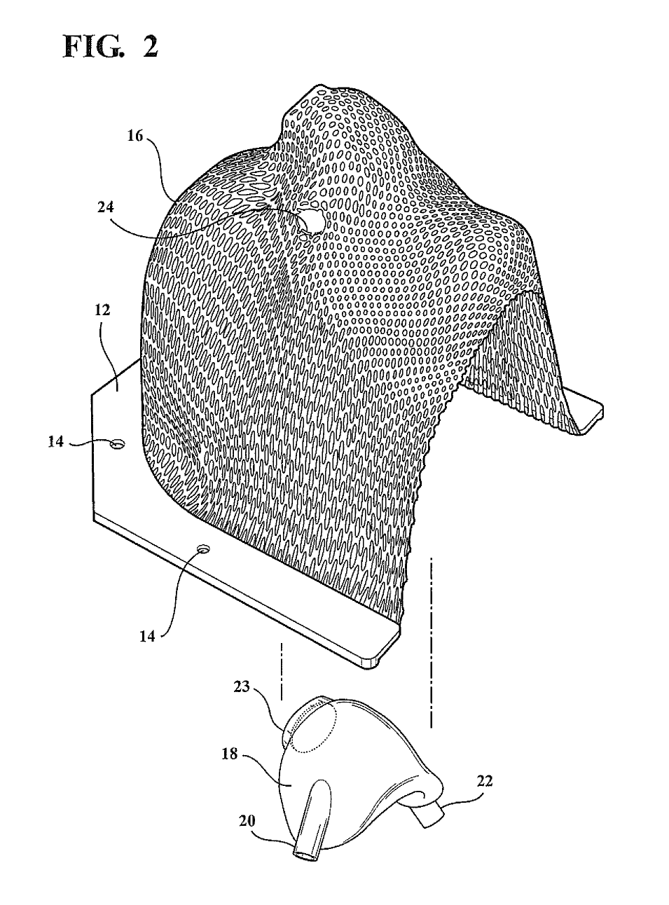

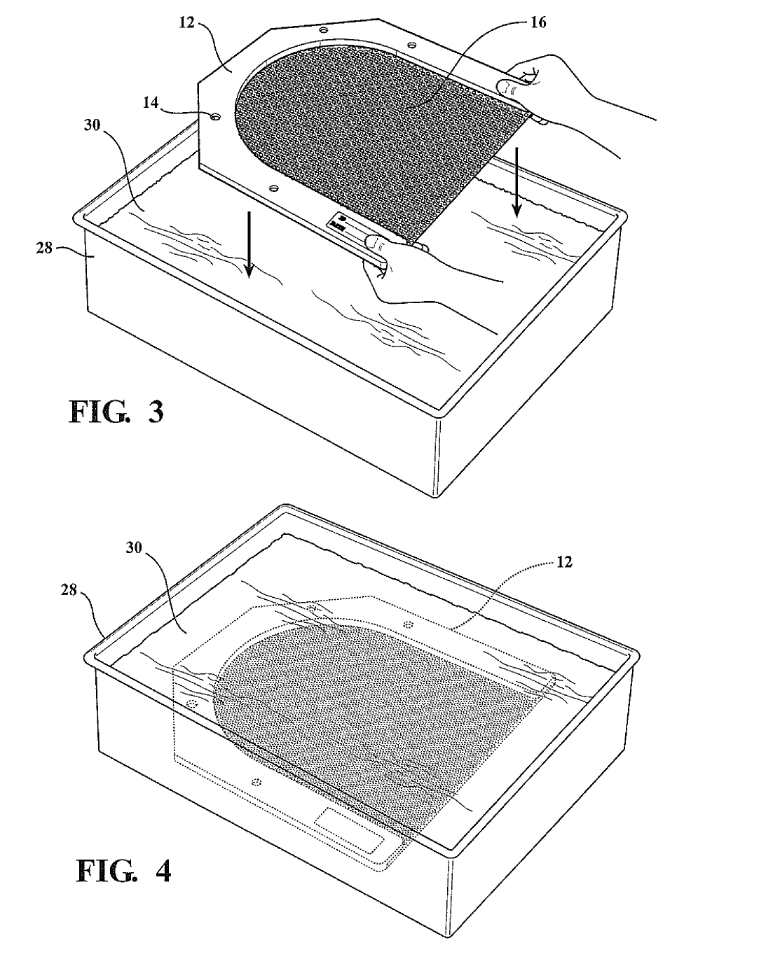

Referring now to FIGS. 1 through 5, there is shown a head immobilizing device 10 in the form of a facially contoured net-like mask 16 attached to a U-shaped rigid frame 12 with holes 14 to permit the device 10 to be secured to the surface of a treatment table or chair. In FIGS. 1 and 2, the device 10 comprises a net 16 of heat-softenable, perforate, thermoplastic sheet material which has been conformed to a patient's facial contours so as to secure the patient's head against movement during a radiation procedure. The mask sheet, prior to the contouring/conforming step, has a pattern of holes of approximately 2 mm. in diameter over substantially its entire area and a thickness of between 2 and 3 mm. The pre-conforming configuration is shown in FIG. 3 and hereinafter described in greater detail. After the conforming or contouring steps are performed, most of the holes in the net are necessarily stretched into a longer and occasionally wider size and the material becomes thinner as a result of the increased area. A suitable mask 10 is available from Klarity Medical Products and is described in Klarity's “Radiation Product Catalog” along with a number of other products. Masks of other designs are used with different tables and/or chairs available from multiple sources. Full upper body immobilizers using a similar construction are commercially available and our inventions contemplate the use of such devices as well.

In the embodiment of FIG. 1, the mask 16 has been conformed not only to the contours of a patient's face and head, but also to a soft elastomeric inhaler mask 18 having short tubes 20 and 22 communicating to the interior volume thereof, as well as a center outlet 23 which may have a check valve. A suitable inhaler mask is available from Accutron, Inc. as stated above and is more fully described in U.S. Pat. No. 3,109,839, the disclosure of which is incorporated herein in full. The inhaler is pre-shaped to sealingly conform to a person's face, over the nose, and to define an interior volume or cavity. The mask net 16 is altered after configuring to provide holes for the tubes 20, 22 of the inhaler 18 such that the inhaler fits within the contours of the fully contoured mask net 16 with tubes 20, 22 protruding as shown in FIGS. 1 and 2.

Because the resulting mask 10 is used through a series of oncological treatments, such as radiation therapy, and is personal to the patient, a label 26 is applied to the frame 12 so that the mask 10 associated with a particular patient can be readily identified and used in all of the treatments in the series.

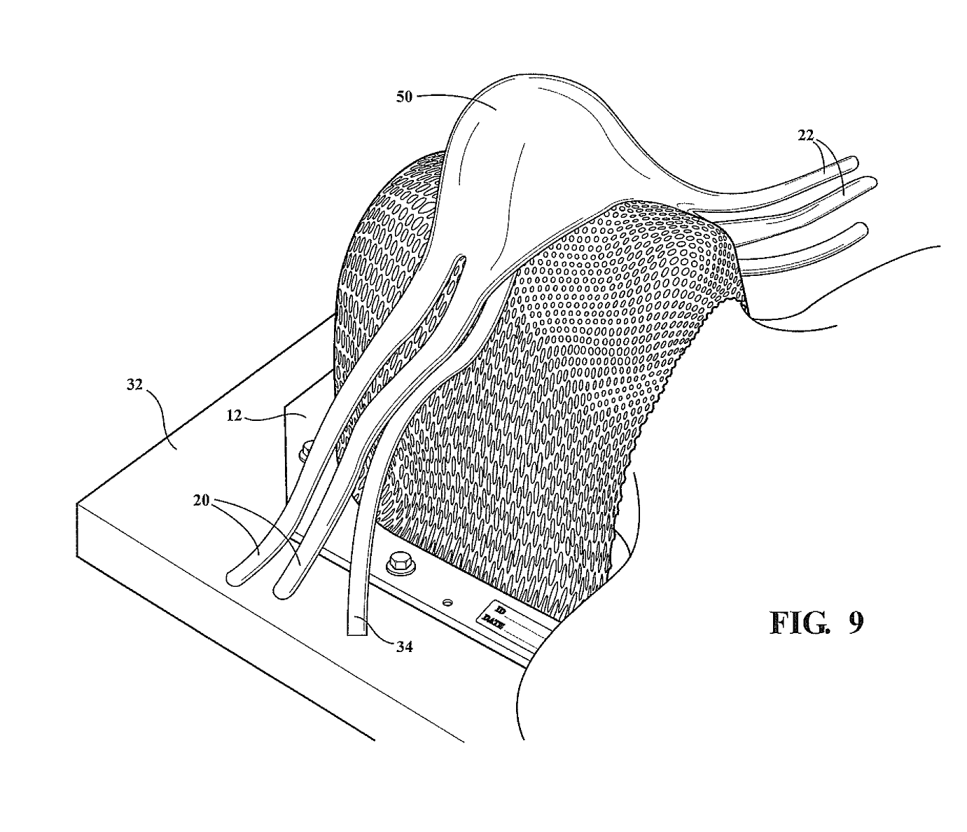

The tubes 20, 22 allow the device 10 with the inhaler 18 to be used in various ways. If the room in which the procedure is carried out is well ventilated, both tubes 20, 22 may supply the anesthetic to the inhaler while the outlet 23 serves to exhaust the gaseous anesthetic as well as the patient's exhalant. Alternatively, one tube 20 may be used as a supply and the other tube 22 as an exhaust. A still further alternative is to use both tubes 20, 22 as supply tubes and add a vacuum-assisted scavenging device to clear the treatment room of anesthetic as shown in FIG. 9. In any case, the pressure of the nitrous oxide supply is too low to operate the check valve in the outlet 23. However, the pressure added by the patient's exhalation does open the valve and exhaust the patient's breath.

### The Method of Preparing the Device

As described above, an aspect of the disclosure is provided herewith is the manner in which the completed and conformed device 10 in association with the inhaler 18 is prepared. Referring to FIGS. 3 through 8, the steps of the preparation process are hereinafter described.

The mask frame 12 with a perforated, flat uncontoured thermoplastic net 16 secured thereto is first immersed in a tray 28 containing a heated fluid or fluidized material 30 so as to soften the thermoplastic net 16 to the point where it is readily conformed without being hot enough to create pain or injury to a patient. As shown in FIG. 4, the mask 10 comprising the frame 12 and net 16 is fully immersed in the heated and fluidized material 30 and is allowed to remain in the material long enough to become soft and conformable.

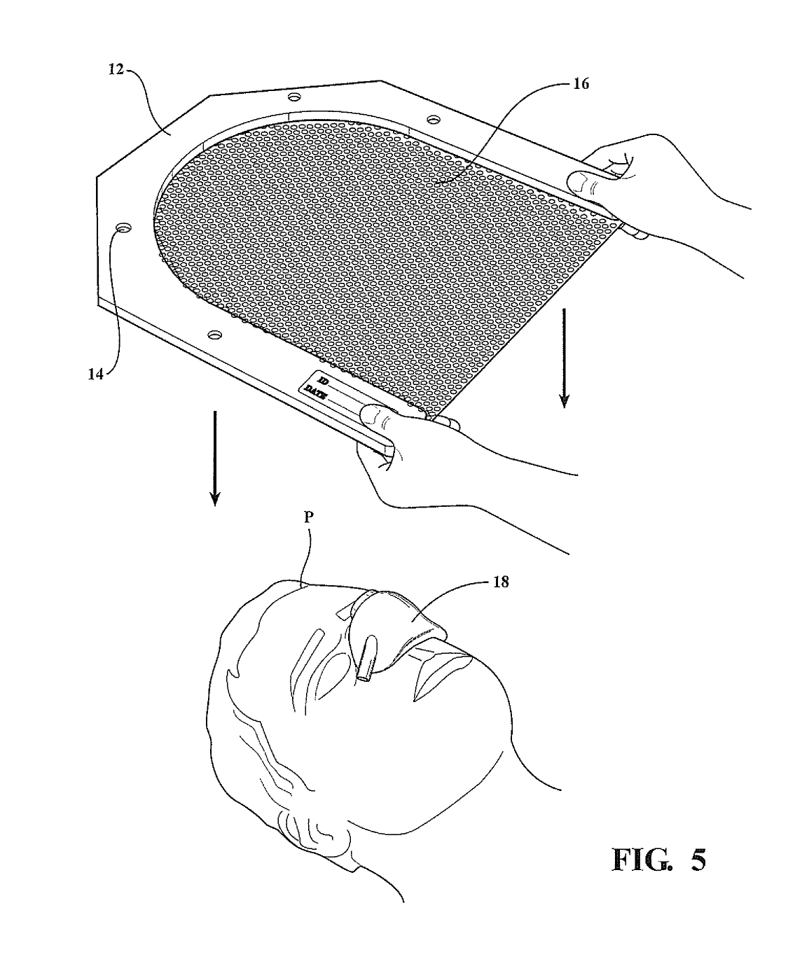

As shown in FIG. 5, the next step is to place the inhaler 18 over the nose of the patient P or a model representing the patient with sufficient accuracy and thereafter placing the frame 12 with the heat softened net 16 over the patient P with the inhaler 18 in place. The device 10 is gradually moved toward the treatment surface 32 until the frame makes full contact with the surface 32. At this point, the net is fully contoured. The net 16 may then be removed from the patient or model and the contours remain permanent.

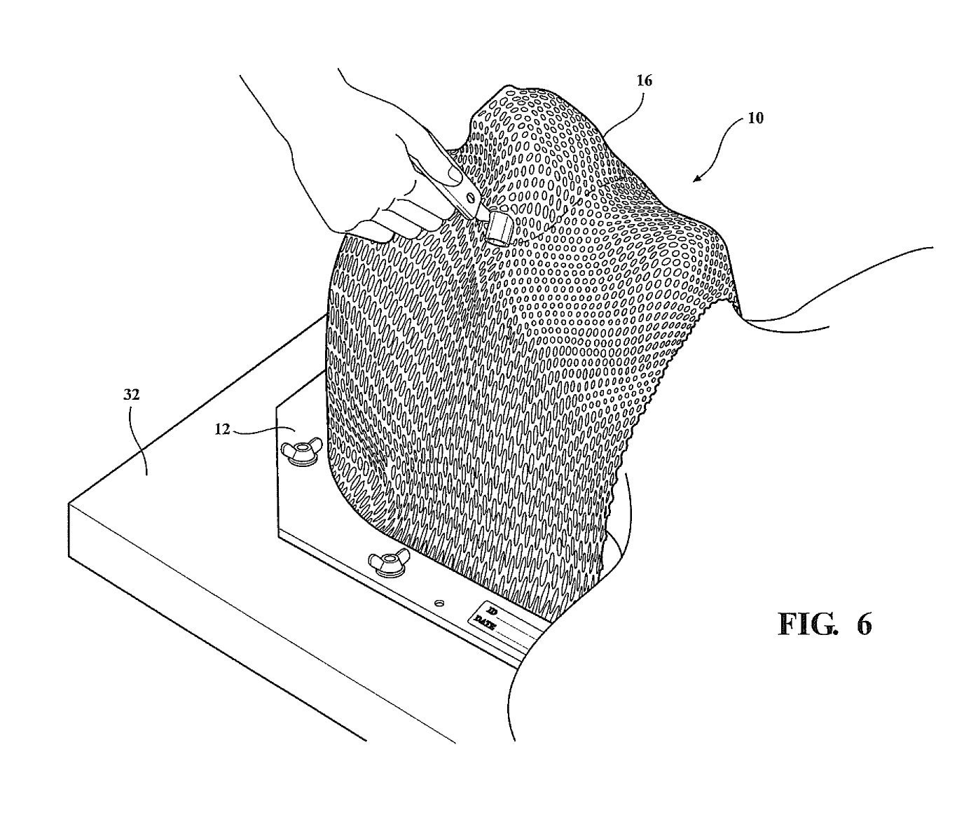

The result is shown in FIG. 6, wherein the mask net 16 has been fully conformed to the patient's face and head and the frame 18 is ready to be bolted or screwed down to the surface of a treatment table 32 or the like. A technician uses a small tool to create openings in the net 16 for the tubes 20, 22 as previously described. The outlet 23 of the inhaler 18 has sufficient clearance through the original holes to operate satisfactorily so no further enlargement of the holes or cutting of the net 16 is generally necessary. The molding or contouring of the thermoplastic net 16 and the extension of the tubes 20, 22 through the mask after contouring is generally sufficient to hold the inhaler 18 to the net 16 during handling and use. They may, however, be separated for sterilization or replacement of the inhaler if deemed necessary.

### The Treatment Process

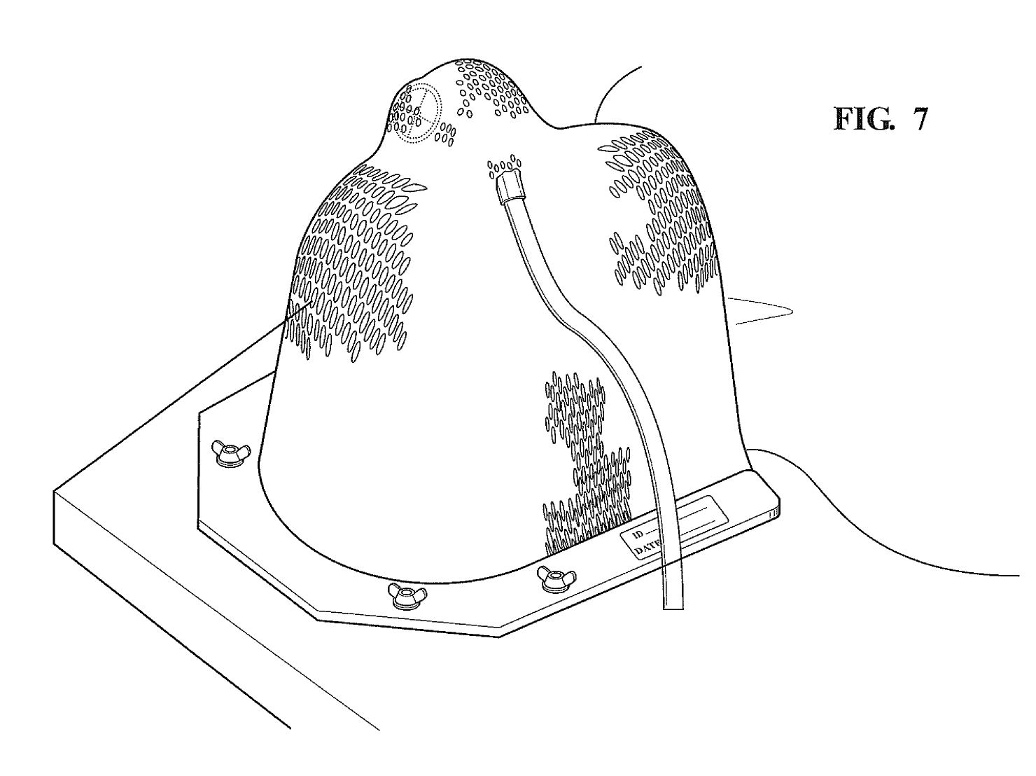

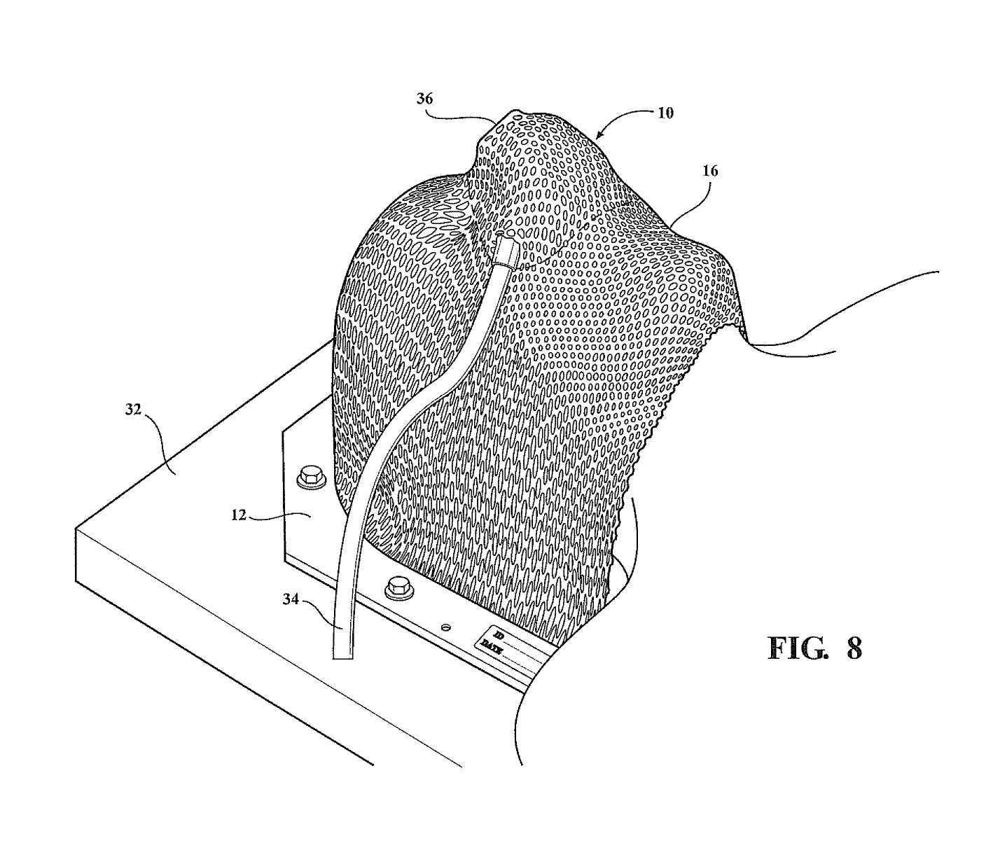

Referring to FIGS. 7 and 8, the treatment process involves mounting the mask/inhaler on the patient and securing it to the surface 32 of the treatment table. The next step is for a technician to connect supply tubes 34 to the inhaler so as to cause the flow of an anesthetic or sedative in gaseous form to the patient P before starting the oncological treatment. Although only one inlet or supply tube 34 is shown in FIG. 7, it is to be understood that a second tube may be similarly connected to the short inhaler tube 22 or that the second tube may be used as an exhaust. Once the flow of anesthetic or sedative in gaseous form has started, the result is to generally calm the patient and relieve stress and anxiety generally associated with the treatment process and the immobilization.

### An Alternative Embodiment

FIG. 9 illustrates an alternative manner in which the device 10 can be used. In this arrangement, the device 10 is constructed as shown in FIGS. 1 through 8 with the inhaler 18 placed over the patient's nose and both tubes 20, 22 connected to a nitrous oxide supply. In addition, a scavenging mask 50 is placed over the mask 16 so that it covers the inhaler outlet 23. The scavenging mask is connected to an exhaust outlet to collect and dispense the excess and exhaled nitrous oxide so it does not affect the attending medical staff in the room with the patient. The scavenging mask may be of the type offered for sale by Porter Instruments; see www.porterinstrument.com.

By way of summary, this disclosure essentially covers three topics: first, the configuration of the device 10 with the inhaler 18 in association therewith; second, the manner in which the combination of the immobilizing mask and the inhaler are associated with one another for use in the treatment process and; third, the treatment process itself wherein the head is immobilized, the anesthetic or sedative in gaseous form is administered to the patient through the inhaler 18 and the oncological treatment is carried out as prescribed.

It will be understood that various changes and additions to the subject matter described herein can be made without departing from the general purpose of the disclosure and the apparatus described herein. The Kiarity™ mask is given in this disclosure by way of example and not by way of limitation. Similarly, the Accutron™ inhaler and the Porter scavenger mask are also given by way of example and not by way of limitation, notwithstanding that these products have both been found to be well-suited for the purposes and end result to be achieved by and through the systems described herein.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A therapy method comprising the steps of:
(a) immobilizing a portion of a patient's body with a body-conforming mask;
(b) administering a gaseous anesthetic to the patient using an inhaler associated with said mask; and
(c) administering therapy to an immobilized area of the patient's body.

2. The method as defined in claim 1 wherein the mask is of the type comprising a moldable, perforate thermoplastic sheet covering a patient's face or neck and a frame for securing the mask to an operating platform.

3. The method as defined in claim 1 wherein the inhaler comprises an elastomeric body configured to provide a cavity which fits sealingly over and around a patient's nose, and ports connected to the body for gaseous fluid flow into and/or out of the cavity.

4. An apparatus for immobilizing a patient's upper body during a treatment comprising:
a frame;
a moldable thermoplastic sheet attached to the frame that can be conformed to a patient's body contours; and
an anesthetic inhaler attached to the conformed sheet in a position chosen to cover the patient's nose when the apparatus is in use; said inhaler having at least one gas inlet port.

5. An apparatus as defined in claim 4 wherein the inhaler is made of an elastomeric material.

6. An apparatus as defined in claim 4 wherein the sheet is molded over the inhaler and at least one gas supply tube is attached to said port through said sheet.

7. A method for preparing an immobilizing mask for the head and neck of a patient comprising the step of:
molding a heat-softened frame-bound conformable, perforate plastic sheet over an inhaler placed on a patient's face or a model thereof so as to simultaneously conform the sheet to the inhaler and the patient facial contours.

8. An immobilizing device for a patient undergoing radiation therapy comprising:
a perforate thermoplastic mask which has been contoured to a patient's face and head;
an inhaler configured to fit sealingly over the patient's nose and having a gaseous inlet port and a scavenging port;
said mask and inhaler being mechanically associated with one another to work as a unit during radiation therapy; and
means for securing the mask and inhaler as a unit to a treatment table.
